(19) United States
(12) Patent Application Publication
(10) Pub. No.: US 2010/0248272 A1
(43) Pub. Date: Sep. 30, 2010

(54) Method for identifying Smk box riboswitch modulating compounds

(75) Inventor: Ailong Ke, Ithaca, NY (US)

Correspondence Address: SCULLY SCOTT MURPHY & PRESSER, PC, 400 GARDEN CITY PLAZA, SUITE 300, GARDEN CITY, NY, 11530, US

(73) Assignee: Cornell University, Ithaca, NY (US)

(21) Appl. No.: 12/798,214

(22) Filed: Mar. 31, 2010

Related U.S. Application Data
(60) Provisional application No. 61/165,259, filed on Mar. 31, 2009.

(51) Int. Cl. G01N 33/53; G06G 7/58; C07D 473/34; C07H 15/04; C12N 1/20; G06F 19/00

(52) U.S. Cl. 435/7.93; 703/11; 544/277; 536/17.2; 435/253.4; 435/252.1; 702/19

(57) ABSTRACT

The invention is directed, inter alia, to a method for identifying a compound that modulates a gene expression of an mRNA molecule containing a S_{MK} box riboswitch that contains a SAM binding pocket, wherein the mRNA molecule is produced by a lactic acid bacteria, the method comprising: (i) generating a database of structural coordinates that includes atomic positions of the S_{MK} box riboswitch and distances of these atomic positions to atomic positions of a first compound complexed to the S_{MK} box riboswitch; (ii) identifying key interactions between the S_{MK} box riboswitch and the first compound from the database of structural coordinates; and (iii) devising a derivative compound of the first compound by use of a computer modeling program, wherein the derivative compound possesses structural modifications, relative to the first compound, that results in an improved modulating interaction between at least one location of the SAM binding pocket and derivative compound.

### CROSS REFERENCE TO RELATED APPLICATION

This application claims the benefit of priority from U.S. Provisional Application No. 61/165,259, filed on Mar. 31, 2009.

### GOVERNMENT SUPPORT

This invention was made with government support under GM079238 awarded by the National Institutes of Health. The government has certain rights in the invention.

### FIELD OF THE INVENTION

The present invention relates generally to methods for identifying compounds with pharmaceutical properties amongst a library of possible compounds, and more particularly, to methods in which computer modeling is included in the method in order to identify such compounds.

### BACKGROUND OF THE INVENTION

Riboswitches are regulatory RNA molecules that recognize specific small molecules (e.g., S-adenosyl methionine, or SAM), and control downstream gene expression at either the transcriptional or translational level. Most riboswitches have been found in Gram-positive bacteria, such as Bacillus anthracis, Staphylococcus, Enterococcus, Streptococcus, Listeria, Clostridium, and Mycobacterium. The riboswitches found in these bacteria belong to the SAM-I or SAM-II classes of riboswitches, both of which have been structurally elucidated in the art. Certain compounds have been identified that can favorably interact with the binding pockets of these riboswitches, thereby modulating the activity (i.e., gene expression activity) of these riboswitches.

However, compounds known to form favorable interactions with SAM-I and SAM-II riboswitches have been found not to form the same favorable interactions with a third type of riboswitch known as the SAM-III (or S_{MK} box) riboswitch. The S_{MK} box riboswitch has only been identified in lactic acid bacteria regulating the translation of SAM synthetase (metK) genes. This group of bacteria includes some well-known virulent pathogens, such as Streptococcus pneumoniae and Enterococcus faecalis.

### SUMMARY OF THE INVENTION

The present invention is directed, inter alfa, to a method for the identification of compounds that target the S_{MK} box riboswitch. In particular embodiments, the method includes identifying a compound that modulates a gene expression of an mRNA molecule containing a S_{MK} box riboswitch that contains a SAM binding pocket.

In further embodiments, the method includes:

(i) generating a database of structural coordinates that includes atomic positions of the S_{MK} box riboswitch and distances of these atomic positions to atomic positions of a first compound complexed to the S_{MK} box riboswitch;

(ii) identifying key interactions between the S_{MK} box riboswitch and the first compound from the database of structural coordinates; and

(iii) devising a derivative compound of the first compound by use of a computer modeling program, wherein the derivative compound possesses structural modifications, relative to the first compound, that results in an improved modulating interaction between at least one location of the SAM binding pocket and derivative compound.

In another embodiment, the invention is directed to a derivative compound found by the above method. The derivative compound is capable of modulating, and in particular embodiments, deactivating or inhibiting, a S_{MK} box riboswitch in order to modulate, deactivate, or inhibit gene expression (i.e., activity) of a lactic acid bacteria.

In another embodiment, the invention is directed to a method for modulating a gene expression of a lactic acid bacteria by contacting the lactic acid bacteria with a derivative compound described herein. In particular embodiments, the lactic acid bacteria is selected from Streptococcus pneumoniae and Enterococcus faecalis.

In yet another embodiment, the invention is directed to a method for identifying a riboswitch having a set of structural features in a SAM binding pocket therein that are common with structural features found in a S_{MK} box riboswitch SAM binding pocket for which a database of structural coordinates is provided. The method includes entering structural data of the S_{MK} box riboswitch and structural data of at least one riboswitch of interest into a computer database, and instructing the computer to identify those riboswitches that have structural features in common with the structural features of said S_{MK} box riboswitch SAM binding pocket.

### BRIEF DESCRIPTION OF THE FIGURES

The file of this patent contains at least one drawing executed in color. Copies of this patent with color drawing(s) will be provided by the Patent and Trademark Office upon request and payment of the necessary fee.

FIGS. 1A, 1B, 1C. Structure of a S_{MK} box riboswitch. (A) Secondary structure of the S_{MK} box riboswitch based on phylogenetic analysis. Black capital letters, residues that are 100% conserved; blue, 50-85% conserved; R, G or A; W, A or U; Y, C or U; thick dotted lines, hypervariable loops; solid ladders, conserved secondary structure; gray and pink shaded areas, SD sequence and AUG start codon, respectively. (B) Secondary structure of the S_{MK}6 riboswitch RNA based on the crystal structure. Helices P1 through P4 are colored in cyan, green, silver and yellow, respectively. Gray shading, SD sequence; solid magenta lines, direct contacts between the RNA and the SAM molecule; dashed magenta lines, tertiary interactions between J3/2 and P2 and J2/4. (C) Cartoon representation of the crystal structure of the SMK riboswitch. SAM is shown in overlapping CPK and surface representations in magenta and silver, respectively. The coloring scheme for the RNA is consistent with b.

FIGS. 2A, 2B. Stereoview depictions of the SAM binding pocket and key interactions between the S_{MK} box riboswitch and SAM.

FIGS. 3A-3E. Enlarged views of selected SAM-S_{MK} box riboswitch interactions.

FIGS. 4A-4B. Drawings showing some of the possible locations of the SAM template that can be appropriately functionalized to optimize interactions between the derivatized SAM molecule and the SAM binding pocket of the S_{MK} box riboswitch. (A) Some key locations for derivatization on the SAM molecule. (B) Besides shortening of methionine tail, sulfonium ion (S⁺) can be replaced with Te⁺, Se⁺, or N⁺, and/or C2 functionalization with a group X, which can be, for example, a halogen (e.g., F) or amino group (e.g., —NH₂).

FIG. 5. Flow diagram summarizing the Systematic Evolution of Ligands by Exponential Enrichment (SELEX) experiment as used herein for finding structurally analogous riboswitches from a library of riboswitches.

FIGS. 6A, 6B. Effects of S_{MK} box riboswitch mutagenesis on SAM binding and apparent SAM binding constant (Kₐ) determination. (A) Effects of mutation (with s.d.) on SAM binding by size-exclusion filtration assay, as described. (B) SAM and SAH K_{d} determination by competition binding assay. Binding curves are shown for the full-length E. faecalis metK RNA (open diamonds, K_{d}=0.85 μM) and the S_{MK}6 crystallization construct (filled diamonds, K_{d}=0.57 μM). Unlabeled SAH showed no competition at a >100-fold excess of SAH over SAM.

FIG. 7. Stereoviews of the binding pocket in the S_{MK} box riboswitch in complex with Se-SAM. The location of the selenium atom is confirmed by the strong anomalous-difference density shown in blue contoured at 8 σ. The rest of the binding pocket in the Se-SAM-bound S_{MK} structure is almost identical to that in the SAM-bound structure. Magenta mesh signifies the simulated composite omit electron-density map of Se-SAM contoured at 1.5 σ.

FIG. 8. Stereoviews of the binding pocket in the S_{MK} box riboswitch in complex with SAH from a direction similar to that shown in FIG. 7. The simulated annealing omit map contoured at 0.8 σ level clearly shows that the ribose and sulfide moieties rotate 180° to exit the RNA from the linker helix side.

FIGS. 9A, 9B, Drawings comparing the conformations that occur with SAM and SAH molecules in various riboswitch and protein structures. (A) SAM conformation from all three classes of SAM riboswitches (i.e., SAM-I, SAM-II, and SAM-III), aligned along the ribose portion. Despite conformational differences in the adenosine base and the methionine tail, the sulfur atom makes a constant 3.0 Å polar interaction with the O4′ of the ribose. (B) Comparison of the O4′-C4′-C5′-S dihedral angle within the SAM and SAH molecules. Molecules are aligned along the O4′-C4′-C5′ linkage. Gauche conformation about the C4′-C5′ bonds (labeled S^{SAM}) is the predominant conformation of SAM in the riboswitch complexes (the CS'-S bond is colored in yellow, cyan, and magenta for the S box (SAM-I), SAM-II and S_{MK} box (SAM-III), respectively). By contrast, a survey of 30 randomly chosen SAH-bound protein structures revealed that in 87% of the structures, the sulfur (SSAH-protein) adopts the anti conformation (pointing towards the 6 o'clock position). SAH in the S_{MK} box structure adopts a near anti conformation (the sulfur atom is labeled S^{SAH} and colored in orange).

FIGS. 10A-10C. Stereoviews showing the metal binding sites in the S_{MK} box RNA. (A) Overall view of the fifteen strontium binding sites on the S_{MK} box RNA. (B, C). Electron density map (gray mesh) showing two strontium ions (Sr208 and Sr207) bound to their respective sites on the S_{MK} box RNA. The magenta mesh shows the simulated composite omit electron density map of strontium contoured at 1σ. Yellow ball, strontium ion; red ball, water. The coloring scheme for the RNA is consistent with that in FIGS. 1A-1C.

FIG. 11. Graph showing 2-AP fluorescence quenching in response to SAM. Increasing concentrations of SAM were added to bipartite metK leader RNA containing an internal 2-AP modification at residue A29. 2-AP fluorescence at steady-state was measured at 375 nm with an excitation wavelength of 310 nm. Increasing SAM resulted in quenching of 2-AP fluorescence for the wild-type RNA (open diamonds), but no substantial change in fluorescence was observed with a U72C mutant RNA (closed diamonds) that is predicted to bind SAM very poorly. Percent quenching (ΔF/F₀×100) represents the change in fluorescence normalized to the initial fluorescence observed in the absence of SAM. Data were analyzed by nonlinear regression analysis to determine an apparent K_{d} of 1.0 μM for the wild-type RNA.

### DETAILED DESCRIPTION OF THE INVENTION

In a first aspect, the invention is directed to a method for identifying a compound that modulates a gene expression of an mRNA molecule containing a S_{MK} box riboswitch that contains a SAM binding pocket. Such mRNA molecules are thus far known to be produced only in lactic acid bacteria.

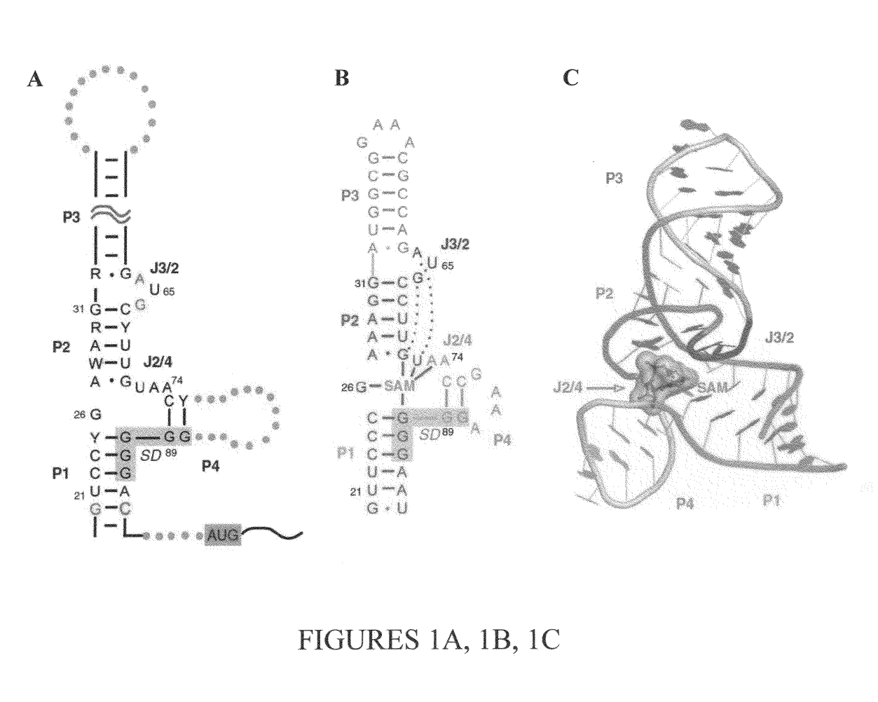

The S_{MK} box riboswitch (FIGS. 1A-1C) regulates the translation of SAM synthetase (metK) genes in lactic acid bacteria by sequestration of the Shine-Dalgarno (SD) sequence, which is essential for loading of the 30S ribosomal subunit for translation initiation. Notably, the SD sequence within the S_{MK} box family (GGGGG) differs from the consensus SD sequence (GGAGG) at the central position (FIG. 1). The three Gs at the 3′ portion of the SD sequence, in conjunction with the following two nucleotides, base pair with the anti-Shine-Dalgarno (ASD) sequence in the presence of SAM, which hinders the binding of 30S ribosomal subunits to the mRNA. Mutational studies have shown that the S_{MK} box riboswitch differs from most metabolite binding riboswitches in that the SD-ASD pairing is required for SAM binding, indicating that the output domain (the SD-ASD pairing) is an intrinsic part of the ligand-binding domain. In comparing the structures of S_{MK} box riboswitches, they have been found overall to adopt very similar structures, particularly at the SAM-binding region. However, some distinctions in structure can generally be found in peripheral regions.

As understood for riboswitches in general, once a compound complexes to the riboswitch, the compound interacts with the aptamer domain (i.e., sensing portion) of the riboswitch. This interaction is then transmitted to the output domain of the riboswitch, which results in conformational changes of the mRNA molecule that affect gene expression. The gene expression being modulated can be any gene expression regulated by the riboswitch-containing mRNA. However, typically, the gene expression refers to transcription and/or translation processes regulated by the riboswitch-containing mRNA.

The term “modulating” as used herein refers to any change (i.e., modification) in gene expression of the mRNA molecule caused by interaction of the compound with the riboswitch-containing mRNA. Typically, the modulation is an attenuation of a gene expression, which furthermore includes, for example, a deactivation or inhibition of the gene expression. However, in some embodiments, the modulation is an activation of a gene expression, as found in the S-adenosylhomocysteine (SAH) sensing riboswitches (Mol. Cell., 2008 Mar. 28; 29(6):691-702).

In the method for identifying a modulating compound, a database of structural coordinates is first provided (i.e., inputted into a computer) for the S_{MK} box riboswitch complexed to a first compound. As used herein, the “first compound” functions as a “template compound” or “starter compound”, i.e., a compound that functions as a starting point (i.e., as a template structure) in seeking a derivative thereof that is a more effective modulating compound.

The database of structural coordinates includes any of the structural information typically gathered from an x-ray crystallography analysis, including atomic positions of the S_{MK} box riboswitch and distances of these atomic positions to atomic positions of the first compound complexed to the S_{MK} box riboswitch. As used herein, “distances” can also include additional information, such as orientation between atoms or molecular groups, which may further include information on molecular angles, packing arrangements, types of interactions (including long-range interactions), conformations, and the like.

As would be appreciated by those in the art, the original derivation of a database of structural coordinates first entails obtaining quality crystals of the S_{MK} box riboswitch complexed to the first compound so that an x-ray crystal structure analysis can be performed. As described in the Examples of this application, the first structure determination of a S_{MK} box riboswitch-compound complex has been achieved in accordance with the present invention.

After the database of structural coordinates has been generated and entered into a computer, key interactions are identified between the S_{MK} box riboswitch and the first compound from said database of structural coordinates by appropriate computational methods. The key interactions generally include at least one atomic (or chemical group) position within the SAM binding pocket of the S_{MK} box riboswitch that interacts with the first compound. The interaction is typically evidenced by a bonding interaction, such as a van der Waals, hydrogen bond, ionic, or even covalent interaction. As known in the art, the different types of interactions are generally characterized and distinguished by the atom-atom distance of the interaction.

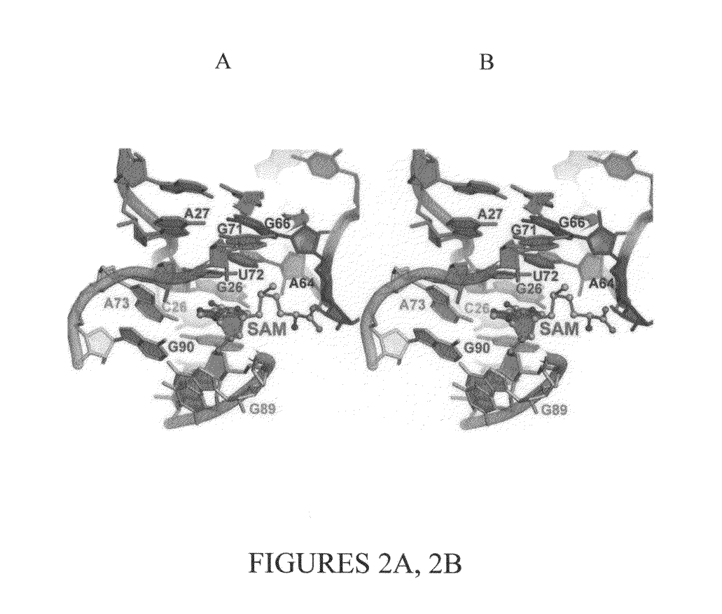

FIGS. 2A and 2B are stereoviews showing the SAM binding pocket and key interactions between the S_{MK} box riboswitch and SAM. The labeling and base coloring scheme are consistent with that in FIG. 1. As particularly shown from FIGS. 2A and 2B, the adenosine moiety of SAM is shown to base-stack between U72 and G90.

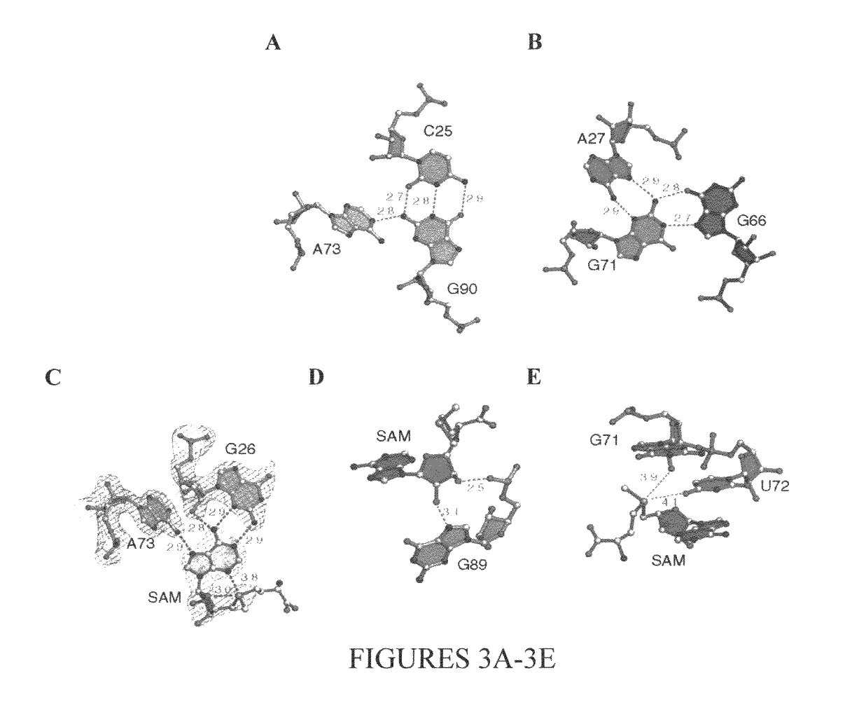

FIGS. 3A-3E show particular key interactions in further detail. FIG. 3A depicts how the A73•G90-C25 base triple paves the ‘floor’ of the SAM binding pocket. The C-G base pair is co-planar, whereas A73 contacts from the minor groove of G90 at a 451 tilted angle, which orients N6 of A73 for SAM recognition one base plane above. FIG. 3B depicts how the A27•G71•G66 base triple defines the ‘ceiling’ of the binding pocket, where the sheared A27•G71 base pair in P2 is contacted at the major groove side by G66 of the J3/2 bulge. FIG. 3C depicts recognition of the adenosine base of SAM overlapped with the 12 Å experimental electron-density contoured at 1.5 σ. Atoms beyond sulfur in SAM do not have corresponding electron density, thereby indicating disorder. The adenine of SAM is extensively recognized through hydrogen-bond interactions from A73 and G26. SAM has two intramolecular electrostatic interactions from the sulfonium ion to the O4′ and N3 of SAM (gray mesh, RNA density; orange mesh, SAM density). FIG. 3D shows that the 3′-hydroxyl group of the ribose of SAM is recognized by a hydrogen bond to the phosphoryl oxygen of G89, whereas the 2′-OH makes a rare π-hydrogen bond interaction with the N7 of G89. FIG. 3E shows that the positively charged sulfonium ion of SAM makes favorable electrostatic interactions with O4 of U72 and the 2′-hydroxyl group of G71. Distances are given in Angstroms. Carbon, oxygen, nitrogen, sulfur and phosphorus atoms are colored gray, red, blue, yellow and orange, respectively.

In particular embodiments, the key interaction being considered herein includes at least one atomic position selected from the binding pocket of the S_{MK} box riboswitch. In particular embodiments, the key interaction being considered herein includes at least one nucleotide position selected from U72, G90, A73, G90, C25, A27, G71, G66, G26, G89, and G71 of the S_{MK} box riboswitch. Among these, the invariably conserved G26, U72, A73, and G90 are considered particularly crucial for SAM-binding.

The one or more key interactions are used, by appropriate computer modeling methods, to refine the structural features of the first compound in order to produce a new model compound (i.e., a derivative compound of the first compound) which possesses structural modifications, relative to the first compound, that results in an improved modulating interaction between at least one location of the SAM binding pocket and derivative compound. The term “modulating interaction”, as used herein, refers to an interaction between the derivative compound and S_{MK} box riboswitch which results, or is predicted to result, in a modulation (i.e., modification, adjustment, change, or optimization) of the gene expression of the S_{MK} box riboswitch. The term “improved modulating interaction”, as used herein, refers to an interaction between the derivative compound and S_{MK} box riboswitch, which results, or is predicted to result, in a modulation (i.e., modification, adjustment, change, or optimization) of the gene expression of the S_{MK} box riboswitch that is improved relative to the modulation ability of the first (non-derivative) compound. The improved modulating interaction can be evidenced by a greater modulating effect on the S_{MK} box riboswitch. The greater modulating effect can be evidenced by, for example, a greater deactivation of, greater inhibition of, or more pronounced change in, the gene expression of the S_{MK} box riboswitch (as evidenced by, for example, the results of an assay).

Typically, the improved modulating interaction is predicted by an improved binding (i.e., complexing) interaction between the derivative compound and one or more locations of the SAM binding pocket. Hence, an improved modulating interaction can be predicted by computer modeling adjustment or optimization of one or more variables indicative of an increased binding interaction. For example, by methods well known in the art, the computer modeling process can adjust or optimize a minimum interaction energy, binding energy (i.e., binding constant), and/or dissociation constant between at least one location of the SAM binding pocket and the derivative compound. In some embodiments, a derivative compound is devised which would strengthen some or all of the known interactions as compared to the interactions provided by the first compound. In other embodiments, a derivative compound is devised which would strengthen certain interactions while weakening other interactions as compared to the interactions provided by the first compound. In other embodiments, a derivative compound is devised which would weaken or strengthen one, two, or three particular interactions while leaving the remaining interactions relatively unchanged as compared to the interactions provided by the first compound.

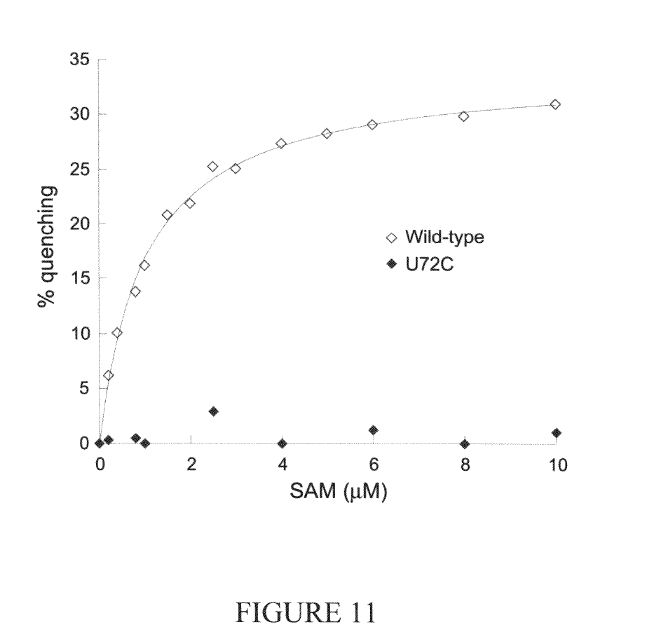

If desired, the modulating ability of the derivative compound can be verified by performing an appropriate assay on the derivative compound. Of course, in order to study the derivative compound by an assay, the derivative compound would need to be obtained either by a commercial source (if available) or by synthesis. In one embodiment, the assay is a competition assay between the derivative compound and at least one compound of known binding interaction with the S_{MK} box riboswitch. In another embodiment, the assay uses a fluorophore-tagged derivative compound in a concentration-dependent fluorescence assay to determine or verify a dissociation constant indicative of the binding energy between the derivative compound and S_{MK} box riboswitch. In yet another embodiment, the riboswitch is fluorescently labeled at strategic positions, e.g., A29, as described in FIG. 11, to sense ligand-binding induced conformational chages, and the dissociation constant is measured from changes in fluorescence. Additionally, the dissociation constant can be derived from thermodynamic changes induced by the binding of the derivative compound measured by isothermal titration calorimetry assays.

In other embodiments, key interactions are verified, or their properties further elucidated, by structural analysis of the S_{MK} box riboswitch-derivative compound complex. In one embodiment, crystals of the S_{MK} box riboswitch-derivative compound complex are obtained, and the resulting crystals analyzed by x-ray crystallography. The structural data obtained therefrom can then be analyzed to determine if key interactions have been modified in a desired manner for improving the modulating effect of the derivative compound relative to the first compound. If desired, the crystallographic structure database of the riboswitch-derivative compound complex can be used as a new (or transitional) template from which a further derivatized compound (i.e., second derivative compound) can be devised in the same manner that the first derivative compound was derived from the first compound. Therefore, by the foregoing embodiment, a second derivative compound can be devised which possesses an improved modulating ability as compared to the first derivative compound. By methods discussed above for the first derivative compound, if desired, the modulating ability of the second derivative compound can be measured by use of an appropriate assay. The structural refinement process can be repeated as many times as desired, e.g., by obtaining a crystallographic structure of the riboswitch-(second derivative compound) complex, and performing computer modeling calculations on the second derivative compound to determine refinements of the structure that would produce an improved modulating compound (i.e., a third derivative compound).

The riboswitch-derivative compound complex can also be studied by spectroscopic analysis to verify key interactions or further elucidate their properties. Some examples of spectroscopic techniques that can be used for this purpose include nuclear magnetic resonance (NMR), infrared (IR), and visible-ultraviolet spectroscopies.

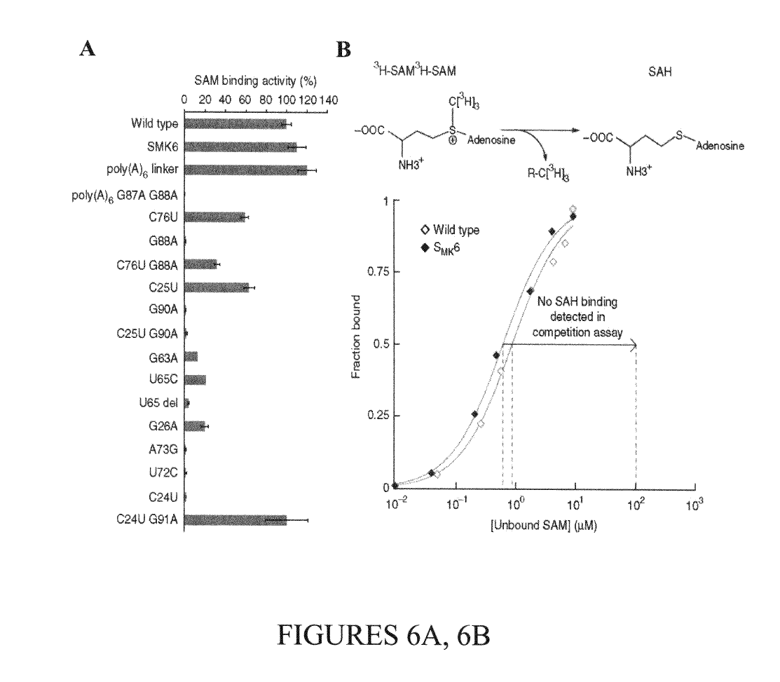

The method can further include other methods for verifying and analyzing key interactions. For example, in some embodiments, key interactions are further elucidated by employing at least one mutation (e.g., scanning mutation) in the SAM binding pocket of the S_{MK} box riboswitch and comparing the binding interaction between the first or derivative compound and mutated S_{MK} box riboswitch. The location and kind of mutation can provide valuable information on specific locations of the SAM binding pocket that are involved in key interactions with the first or derivative compound. Reference is made, for example, to FIGS. 6A and 6B and the discussion thereon.

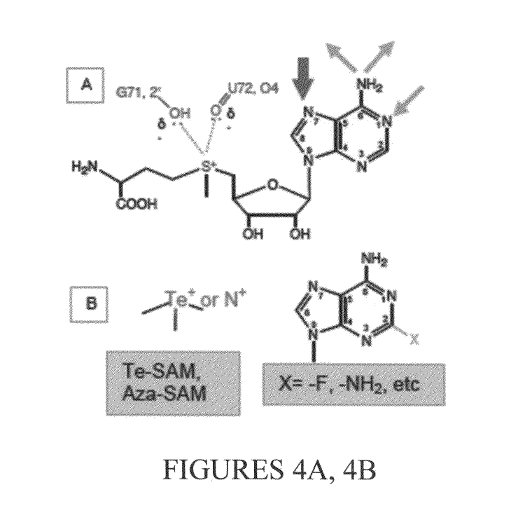

The first compound and any derivative compound can be any compound bearing at least some of the structural features of SAM. Reference is made to FIGS. 4A-4B, which show some of the various substitutions and derivations that can be made on the SAM template structure.

In particular, the first compound and any derivative compound can have a chemical structure encompassed by the following generic structure:

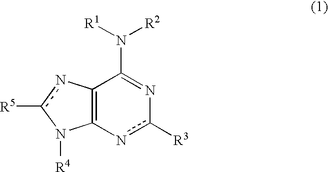

In formula (1), R¹, R², R³, R⁴, and R⁵ are independently selected from hydrogen atom and hydrocarbon groups. The hydrocarbon groups can be straight-chained or branched, cyclic or acyclic, and either saturated or unsaturated. Typically, the hydrocarbon groups considered herein contain at least one, two, or three carbon atoms and up to four, five, six, seven, or eight carbon atoms. Some examples of straight-chained saturated hydrocarbon groups include methyl, ethyl, n-propyl, n-butyl, n-pentyl, n-hexyl, n-heptyl, and n-octyl groups. Some examples of branched saturated hydrocarbon groups include isopropyl, isobutyl, sec-butyl, t-butyl, isopentyl, and neopentyl groups. Some examples of cyclic saturated hydrocarbon groups include cyclopropyl, cyclobutyl, cyclopentyl, cyclohexyl, cycloheptyl, and cyclooctyl groups. Some examples of straight-chained unsaturated groups include vinyl, allyl, 3-buten-1-yl, 2-buten-1-yl, 1,3-butadien-1-yl, acetylenic, and propargyl groups. Some examples of branched unsaturated groups include propen-2-yl, 3-buten-2-yl, 3-buten-3-yl, and 2-buten-2-yl groups. Some examples of cyclic unsaturated groups include cyclopentene, cyclopentadiene, cyclohexene, cyclohexadiene, phenyl, benzyl, diphenyl, tolyl, ethylphenyl, and naphthyl groups. In one embodiment, the hydrocarbon groups are composed solely of carbon and hydrogen. In another embodiment, the hydrocarbon groups can include one, two, three, or more heteroatoms or heteroatom groups. Some example of heteroatoms include oxygen (O), nitrogen (N), sulfur (S), selenium (Se), halogen (e.g., F, Cl, or Br atoms), and phosphorus (P) atoms. Some examples of heteroatom groups include carbonyl (CO), carboxamido (—NHCO—), urea, carboxyl, ester, hydroxyl, ether, amino, and imino groups.

In a first set of embodiments, R¹, R², R³, R⁴, and R⁵ are all hydrogen atoms. In a second set of embodiments, four of R¹, R², R³, R⁴, and R⁵ are hydrogen atoms and one of R¹, R², R³, R⁴, and R⁵ is a hydrocarbon group. In a third set of embodiments, three of R¹, R², R³, R⁴, and R⁵ are hydrogen atoms and the remainder are hydrocarbon groups. In a fourth set of embodiments, two of R¹, R², R³, R⁴, and R⁵ are hydrogen atoms and the remainder are hydrocarbon groups. In a fifth set of embodiments, one of R¹, R², R³, R⁴, and R⁵ is a hydrogen atom and the remainder are hydrocarbon groups. In a sixth set of embodiments, R¹, R², R³, R⁴, and R⁵ are all hydrocarbon groups.

In further embodiments of formula (1), one or both of R³ and R⁵ can additionally be selected from halogen atom, oxo group (O), and amino group (—NR¹R²). The groups R¹ and R² in the foregoing formula of the amino group can be the same or different, and are selected from hydrogen atom and hydrocarbon group as defined above. In one set of embodiments, one or both of R³ and R⁵ are halogen atoms. In another set of embodiments, one or both of R³ and R⁵ are oxo groups. In another set of embodiments, one or both of R³ and R⁵ are amino groups. In another set of embodiments, R³ is a halogen atom while R⁵ is a hydrogen atom, hydrocarbon group, oxo group, or amino group. In another set of embodiments, R⁵ is a halogen atom while R³ is a hydrogen atom, hydrocarbon group, oxo group, or amino group. In another set of embodiments, R³ is an oxo group while R⁵ is a hydrogen atom, hydrocarbon group, halogen atom, or amino group. In another set of embodiments, R⁵ is an oxo group while R³ is a hydrogen atom, hydrocarbon group, halogen atom, or amino group.

The dashed lines in formula (1) indicate the presence or absence of a double bond. It is understood that, according to the rules of chemistry, when R³ is bound to the indicated ring structure via an unsaturated bond (e.g., when R³ is an oxo group), the dashed line adjacent to R³ cannot represent a double bond, but rather, a single bond. However, if R³ is bound to the indicated ring structure via a saturated bond (e.g., when R³ is a hydrogen atom, methyl, ethyl, allyl, phenyl, halogen atom, or amino group), the indicated dashed line adjacent to R³ can represent either a double bond or a single bond. A completely analogous set of rules applies to R⁵.

In particular embodiments, R¹ and R², or R¹ and R³, or R¹ and R⁴, or R¹ and R⁵ are hydrogen atoms, and the remaining groups are independently selected from hydrogen atom, hydrocarbon group, or any other group described above. In other particular embodiments, R¹ and R², or R¹ and R³, or R¹ and R⁴, or R¹ and R⁵ are hydrocarbon groups, and the remaining groups are independently selected from hydrogen atom, hydrocarbon group, or any other group described above. In other particular embodiments, R¹, R², and R³, or R¹, R², and R⁴, or R¹, R², and R⁵, or R¹, R³, and R⁴, or R¹, R³, and R⁵, or R³, R⁴, and R⁵, are hydrogen atoms, and the remaining groups are independently selected from hydrogen atom, hydrocarbon group, or any other group described above. In other particular embodiments, R¹, R², and R³, or R¹, R², and R⁴, or R¹, R², and R⁵, or R¹, R³, and R⁴, or R¹, R³, and R⁵, or R³, R⁴, and R⁵, are hydrocarbon groups, and the remaining groups are independently selected from hydrogen atom, hydrocarbon group, or any other group described above. In other particular embodiments, R¹, R², R³, and R⁴, or R¹, R², R³, and R⁵, or R¹, R³, R⁴, and R⁵, are hydrogen atoms, and the remaining groups are independently selected from hydrogen atom, hydrocarbon group, or any other group described above. In other particular embodiments, R¹, R², R³, and R⁴, or R¹, R², R³, and R⁵, or R¹, R³, R⁴, and R⁵, are hydrocarbon groups, and the remaining group is selected from hydrogen atom, hydrocarbon group, or any other group described above.

In further embodiments of formula (1), R⁴ can additionally be selected from a 4-S-(methionyl)ribosyl group and analogs thereof. In particular embodiments, the 4-S-(methionyl)ribosyl groups and analogs thereof are represented by the following structure;

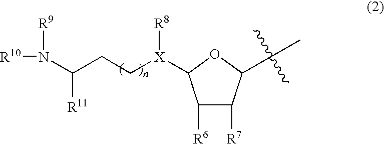

In formula (2), R⁸, R⁹, R¹⁰, and R¹¹ are independently selected from a hydrogen atom and hydrocarbon groups, wherein the hydrocarbon groups are as defined above. The groups R⁶ and R⁷ are independently selected from hydrogen atom and hydroxyl groups. The group X is selected from oxygen atom, sulfur atom, selenium atom, tellurium atom, nitrogen atom, or ammonium group (—⁺NR₄), wherein the four R groups are independently selected from hydrogen atom and hydrocarbon groups as described above. It is understand that, when the group X is selected from oxygen atom, sulfur atom, selenium atom, tellurium atom, or nitrogen atom, these atom groups are necessarily positively charged in the position indicated by X. The subscript n is typically a number of 0, 1, 2, 3, or 4, thereby denoting, respectively, the absence of the indicated methylene group (when n is 0), or a linkage of 1, 2, 3, or 4 of the indicated methylene groups.

In a further embodiment of formula (2), R¹¹ can additionally be selected from a carboxyl-containing or carboxylester-containing group having a structure denoted by, for example, —(CH₂)ₘC(O)OR¹², wherein m is typically 0, 1, 2, 3, 4, 5, or within a range therein, and R¹² is a hydrogen atom or hydrocarbon group as described above. In other embodiments, one or both of the groups R⁶ and R⁷ are selected from ether groups denoted by the formula —OR¹³, where R¹³ is a hydrocarbon group as described above.

In some embodiments, two adjacent groups are capable of interlinking to form a linkage. For example, in some embodiments, R¹ and R² of formula (1) are capable of interlinking to form a cyclic amine structure. In other embodiments, R⁹ and R¹⁰, or R¹⁰ and R¹¹ of formula (2) are capable of interlinking to form a cyclic amine structure. In other embodiments, R³ and R⁴ are capable of interlinking to form a tricyclic fused ring system. The interlinking can result in any type of linkage (e.g., saturated or unsaturated, straight-chained or branched, and either with or without heteroatoms). In particular embodiments, the resulting linkage is a saturated straight-chained or branched linkage, such as a straight-chained or branched alkylene linkage. Some examples of straight-chained alkylene linkages include dimethylene (—CH₂CH₂—), trimethylene, and tetramethylene linkages.

In other embodiments, one or more of R¹, R², R³, R⁴, R⁵, R⁶, R⁷, R⁸, R⁹, R¹⁰, and/or R¹¹ contain reactive portions therein that allow R¹, R², R³, R⁴, R⁵, R⁶, R⁷, R⁸, R⁹, R¹⁰, and/or R¹¹ to form a covalent bond with one or more nucleotides within the SAM binding pocket of the S_{MK} box riboswitch. The reactive portion can include, for example, an activated ester (e.g., succinimide or carbodiimide ester), alkylhalide, carbonyl-containing group (i.e., capable of forming an imide bond), or azide group. In a particular embodiment, a reactive group is included in any of R¹, R², R³, R⁴, and/or R⁵ (and particularly, R³, R⁴, and/or R⁵, and more particularly, R³) to form a covalent bond with nucleotide G26 in the binding pocket of the S_{MK} box riboswitch, or with a nucleotide bound to or engaged in a base-pairing or other interaction with G26, or with a nucleotide one, two, three, four, or five nucleotides from G26, or with a nucleotide within 5, 10, 15, 20, 25, or 30 Å of G26.

In other embodiments, one or more of R¹, R², R³, R⁴, R⁵, R⁶, R⁷, R⁸, R⁹, R¹⁰, and/or R¹¹ (and more particularly, one or more of R¹, R², R³, R⁴, and R⁵, or one or more of R⁶, R⁷, R⁸, R⁹, R¹⁰, and R¹¹) contain a portion therein which engages one or more specific nucleotides in the SAM binding pocket of the S_{MK} box riboswitch in a non-covalent binding interaction. The non-covalent binding interaction can be, for example, a hydrogen bonding, ionic, or van der Waals interaction. To establish a hydrogen bonding interaction, the engaging portion of the group in formula (1) and/or formula (2) can, for example, include a hydrogen acceptor group (e.g., carbonyl- or imino-containing portion) or hydrogen donating group (e.g., amino, hydroxyl, or carboxylic acid group) to effect hydrogen bonding to, respectively, a hydrogen bond donating portion of a nucleotide or a hydrogen accepting portion of a nucleotide.

In some embodiments, a first or subsequent derivative compound includes at least one fluorescent group (i.e., dye or fluorophore group). The fluorescent group can be included, for example, in R¹, R², R³, R⁴, or R⁵ of formula (1), or in R⁶, R⁷, R⁸, R⁹, R¹⁰, or R¹¹ of formula (2). Such a fluorophore-tagged derivative compound can be particularly useful in a fluorescence-based assay in which the derivative compound changes in fluorescence in the presence or absence of a S_{MK} box riboswitch. The fluorophore can be, for example, a fused polycyclic aromatic hydrocarbon (PAH) containing at least two, three, four, five, or six rings (e.g., naphthalene, pyrene, anthracene, chrysene, triphenylene, tetracene, azulene, and phenanthrene), a xanthene derivative (e.g., fluorescein, rhodamine, Oregon green, eosin, and Texas Red), cyanine or its derivatives or subclasses (e.g., streptocyanines, hemicyanines, closed chain cyanines, phycocyanins, and phthalocyanines), naphthalene derivatives (e.g., dansyl and prodan derivatives), coumarin and its derivatives, oxadiazole and its derivatives (e.g., pyridyloxazoles, nitrobenzoxadiazoles, and benzoxadiazoles), pyrene and its derivatives, oxazine and its derivatives (e.g., Nile Red, Nile Blue, and cresyl violet), acridine derivatives (e.g., proflavin, acridine orange, and acridine yellow), arylmethine derivatives (e.g., auramine, crystal violet, and malachite green), and tetrapyrrole derivatives (e.g., porphyrins and bilirubins). Some particular families of dyes considered herein are the Cy® family of dyes (e.g., Cy2, Cy3, Cy3B, Cy3.5, Cy5, Cy5.5, and Cy7), and the Alexa® family of dyes.

In particular embodiments, the first compound is SAM or its near cognate ligand S-adenosyl-L-homocysteine (SAH). The first and subsequent derivative compounds generally exclude SAM or SAH.

Any of the derivative compounds corresponding to formulas (1) and (2) described above can be synthesized by methods known in the art. For example, these compounds can be enzymatically synthesized by SAM synthetase using derivatized adenosine and methionine as substrates, as described, for example, in Biochemistry, 43, 13496-13509 (2004), the contents of which are incorporated herein by reference in their entirety. Alternatively, for example, the derivative compounds can be generated through total synthesis, as described in J. Med. Chem., 52 (5), pp 1388-1407 (2009), the contents of which are incorporated herein by reference in their entirety.

In other aspects, the invention is directed to the derivative compounds, themselves, as well as methods for using the compounds. In particular embodiments, one or more derivative compounds of interest function to deactivate the gene expression of an S_{MK} box riboswitch, and hence, inhibit the growth and functioning of a lactic acid bacteria containing an S_{MK} box riboswitch. In further embodiments, one or more derivative compounds of interest function as antibiotic compounds against a lactic acid bacteria containing an S_{MK} box riboswitch. The deactivation or inhibition of the S_{MK} box riboswitch can result in the inhibition or blocking of any cellular function regulated by the S_{MK} box riboswitch. Some cellular functions that can be inhibited include the transcription and translation processes associated with the mRNA containing the S_{MK} box riboswitch.

The antibiotic activity of the derivative compound can be determined by any of the assays commonly employed for this purpose. The antimicrobial effect is commonly determined by IC₅₀ values, i.e. the minimal concentration of a substance that inhibits growth of microorganisms by 50%, or by MIC values, i.e. the minimal concentration of a substance that completely inhibits growth, both values being determined from toxicity curves. Assays of bacterial growth inhibition can be conducted in liquid media, in which case the IC₅₀ values will be determined from growth curves by a doubling dilution series method in liquid LB (lysogeny broth) medium. Typically, an IC₅₀ value is determined as the concentration required to reduce the growth of an overnight culture to 50% of the control value. Alternatively, a MIC value can be determined from counting colonies grown on Petri dish plates by a doubling dilution series method, where the MIC value is determined as the concentration required to completely inhibit the growth of colonies on Petri dishes.

In another aspect, the invention is directed to a method of modulating a gene expression of a lactic acid bacteria by contacting the lactic acid bacteria with at least one derivative compound described above. In one embodiment, the process is conducted as an in vitro process. The in vitro process of modulating a gene expression of a lactic acid bacteria can be used, for example, as a research tool to determine the efficacy of the derivative compound in modulating the gene expression. In another embodiment, the process is conducted as an in vivo process, e.g., in the treatment of a living organism infected with a strain of lactic acid bacteria. The living organism considered herein is generally mammalian, and more typically, human. Particularly in methods for treatment of a living organism, it is generally desired that the derivative compound is capable of deactivating or inhibiting the growth and/or survival of the lactic acid bacteria.

In yet another aspect, the method is directed to a method for identifying a riboswitch having a set of structural features in a SAM binding pocket therein that are common with structural features found in a S_{MK} box riboswitch SAM binding pocket for which a database of structural features has been obtained (i.e., provided). By having “structural features in common” is generally meant that there is at least about a 75%, 80%, 85%, 90%, 95%, 96%, 97%, 98%, or 99% percent homology between the nucleotides in the SAM binding pocket of a riboswitch of interest and nucleotides in the SAM binding pocket of a S_{MK} box riboswitch. Furthermore, having “structural features in common” can include more than a commonality in nucleotide sequence. For example, the commonality in structural features can also include a commonality in the three-dimensional orientation (i.e., conformations) of the nucleotide sequences. The riboswitch of interest generally considered herein are riboswitches other than SAM-I and SAM-II riboswitches. The riboswitches of interest include, for example, an S_{MK} box riboswitch derived from a source different from the source from where the first S_{MK} box riboswitch was derived, as well as new types of riboswitches that are not classified as either SAM-I, SAM-II or S_{MK} riboswitches. The foregoing discovery process plays an important role in the instant invention by, for example, identifying those riboswitches that can be targeted (i.e., modulated) by one or more of the same derivative compounds, described above, found to effectively target an S_{MK} box riboswitch.

In a first embodiment, the method for finding structurally analogous (i.e., homologous) riboswitches includes entering structural data of a first S_{MK} box riboswitch and structural data of one or more riboswitches of interest into a computer database. The computer is then instructed (i.e., via a suitable computer program) to conduct a comparative analysis of the structural data to identify those riboswitches that have structural features in common with the structural features of the S_{MK} box riboswitch.

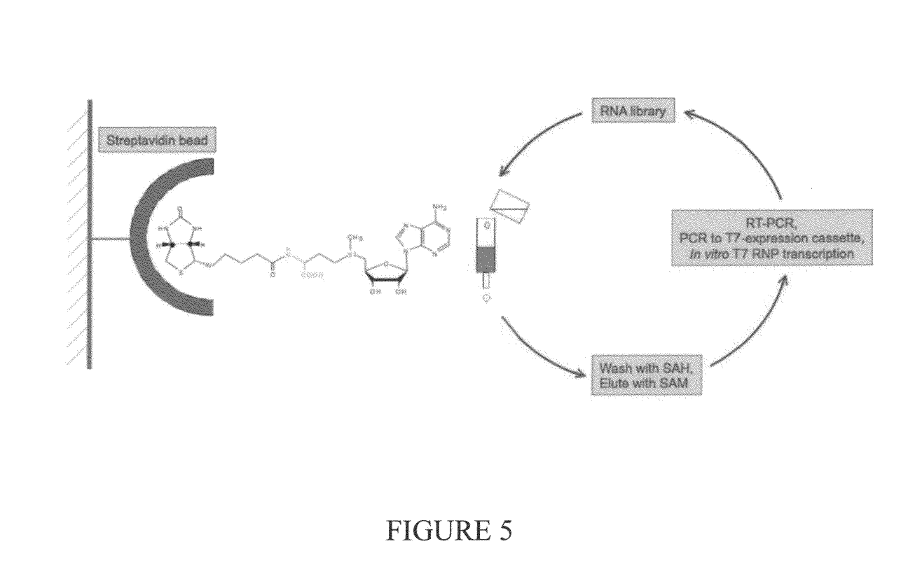

In a second embodiment, the method for finding structurally analogous riboswitches is achieved by experimental testing of a library of riboswitches. A particular example of such an experimental protocol is the Systematic Evolution of Ligands by Exponential Enrichment (SELEX) experiment, the features of which are depicted in FIG. 5. SELEX can be used to search for SAM-binding RNA elements in bacteria, archaeal, or eukaryotic genomes. SELEX includes construction of an RNA aptamer library from genomic DNAs, passing the RNA library through affinity resins where SAM (or an analog thereof) is immobilized through covalent interactions from its methionine tail, then amplifying the bound RNA species using reverse transcriptase by standard procedures. The wash condition may become gradually more stringent in later rounds of SELEX. For example, during final steps of SELEX, the column may be washed with 10 mM SAH to selectively remove aptamers that cannot distinguish SAM from SAH, and those remaining will then be dated using 10 mM SAM for selective amplifications.

Examples have been set forth below for the purpose of illustration and to describe the best mode of the invention at the present time. However, the scope of this invention is not to be in any way limited by the examples set forth herein.

### Example 1

### RNA Preparation and Crystallization

The E. faecalis SMK box riboswitch sequence was inserted into a pUC19 plasmid under the control of a T7 RNA polymerase (RNAP) promoter, with the hepatitis delta virus ribozyme placed at the 3′ end of the S_{MK} box sequence to ensure a homogeneous 3′-end. The crystallization construct initiates 1 nt upstream of the ASD sequence and ends 3 nucleotides (nt) downstream of the SD sequence. In the process of searching for better diffracting RNA crystals, the hypervariable P3 stem was replaced with a five by GC-rich stem capped with a GAAA tetraloop, and the hypervariable linker connecting the SD sequence to the upstream helical domain was systematically shortened. Nine different constructs were generated by combining variations at two hypervariable regions (P3 and P4). Reference is made, for example, to A. Ke, et al., Methods, 34, 408-414 (2004), and R. T. Fuchs, et al., Nat. Struct. Mol. Biol., 13, 226-233 (2006).

To ensure conformational homogeneity, the S_{MK} box RNA was heat-refolded as described in R. T. Fuchs et al., 2008 (Ibid.) and R. T. Fuchs et al., Proc. Natl. Acad. Sci. USA, 104, 4876-4880 (2007). The high-resolution SMK6 crystals grew as extremely slim needle crystals of 15×15 μm² in cross-section from a solution containing 40 mM sodium cacodylate, pH 7.0, 80 mM strontium chloride, 15% (w/v) 2-methylpentane-2,4-diol (MPD) and 2 mM spermine-HCl. The MPD content was raised stepwise to 25% (w/v) before the crystals were flash-frozen in liquid nitrogen.

### Example 2

### Structure Determination

The crystal structure of the E. faecalis S_{MK} box riboswitch was determined. Diffraction data were collected using the microcrystallography setup at beamlines Advanced Photon Source (APS) 24-ID-E and MACCHESS F1 and processed using HKL200042 (Z. Otwinowski, et al., Methods Enzymol., 276, 307-326 (1997)). The initial model was built using COOT from experimental phases calculated from SHELXD and refined using Refmac5 and CNS computer programs. For COOT, reference is made to P. Emsley, et al., Acta Crystallogr. D Biol. Crystallogr., 60, 2126-2132 (2004). For SHELXD, reference is made to G. M. Sheldrick, et al., Acta Crystallogr. A, 64, 112-122 (2008). For Refinae5, reference is made to Collaborative Computational Project, Number 4. The CCP4 suite: programs for protein crystallography. Acta Crystallogr. D Biol. Crystallogr. 50, 760-763 (1994). For CNS, reference is made to A. T. Brunger, et al., Acta Crystallogr. D Biol. Crystallogr., 54, 905-921 (1998) and A. T., Brunger, Nat. Protoc., 2, 2728-2733 (2007).

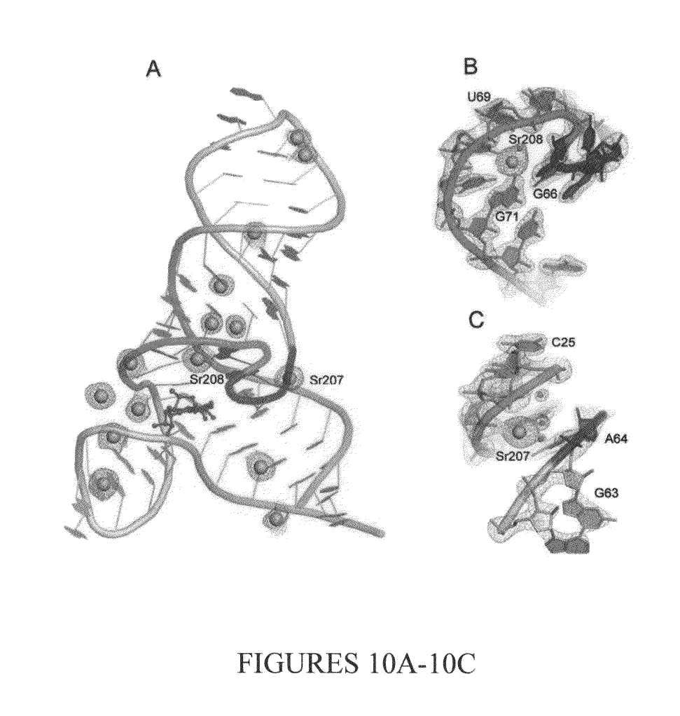

The final model included all nucleotides and a total of 15 strontium ions and 69 water molecules (FIG. 10A-10C, and C. Lu et al., Nature Structural & Molecular Biology, vol 15, no. 10, October 2008, supplementary section). The methionine tail of SAM and the pyrimidine ring of U65 are the only portion of the model without corresponding electron densities. Final R_{work} and R_{free} factors were 22.1% and 22.7%, respectively. Selenium-substituted SAM (Se-SAM) and SAH bound structures were determined by molecular replacement using the PHASER computer program and refined using Refmac5 and CNS computer programs. The optimized version of the S_{MK} box riboswitch is shown in FIGS. 1A-1C (designated as S_{mK}6), and allowed structure determination at 2.2 Å.

### Data Collection and Structure Refinement

A microcrystallography setup was required to obtain high-resolution data sets from the S_{MK}6 needle crystals. Due to very fast radiation decay, the needle crystals were aligned and transferred parallel to the oscillation axis to allow collection of complete data sets with satisfactory redundancy. Data were processed using the HKL2000 computer program.

While most SMK crystals belong to the I4₁22 space group (one molecule per asymmetric unit (ASU)), a few fall into the P4₁2₁2 space group with similar unit cell dimensions (two molecules per ASU). A 3.8 Å single wavelength anomalous diffraction data set was collected from a P4₁2₁2 crystal soaked in 80 mM iridium hexaammine. The initial phase calculated using program SHELXD from eight iridium hexaammine sites was improved by applying a two-fold non-crystallographic symmetry (NCS) to average the two molecules in ASU. The figure of merit was 0.70, and R_{cullis} was 0.57 (0.68 for the outer shell). After solvent-flattening and phase extension to a 2.6 Å native data set, the experimental density map allowed unambiguous model building of 23 nucleotides using program COOT. Twenty nucleotides were missing in the initial model, including the entire P3, J3/2, and part of P2. To locate the P3 helix, an ideal RNA model containing a 5-base pair A-form duplex capped by a GAAA tetraloop was included in the molecular replacement search by using the program PHASER. The correct solution was identified from the top rotation and translation solutions after manual examination of the crystal packing environment. The rest of the missing nucleotides were added one or two bases at a time followed by restrained positional refinement using Refmac5. The SAM molecule was then real-space fitted into the difference map using COOT. The completed structural model was refined against a 2.2 Å 14₁22 native data set in iterative cycles of torsion-angle simulated annealing, group/individual B-factor refinement, and Powell energy minimization in CNS. A final round of TLS refinement in Refmac5 was carried out to model domain movements within the RNA. The same set of excluded reflections was used in free R factor calculation between refinement programs.

### Overall Structure of the S_{MK} Box Riboswitch

The 53-nt S_{MK} box riboswitch RNA folds into an inverted Y-shaped molecule, where helices P1 (SD-ASD helix) and P4 (linker helix) constitute the two short arms, and P3 (top helix) stacks on top of P2 (middle helix) to give rise to the long arm (FIG. 1C). Most of the secondary-structure features match the predictions from the phylogenetic analysis and RNase T1, V1, A and H probing experiments (R. T. Fuchs, et al., 2006 Ibid.), including the SAM-dependent formation of the SD-ASD helix and the protection of nucleotides, including U69, U70, C75 and G88, although the P4 linker helix was not previously assigned. The quintuple-G SD sequence spans the SD-ASD (P1) and linker (P4) helices (FIG. 1B), contributing to the overall folding of the RNA and the SAM binding site. Among the 23 residues in the S_{MK} box riboswitch that are 100% conserved (FIG. 1A), 10 participate in the formation of a pocket inside the three-way junction, where the adenosine moiety of SAM intercalates to allow continuous base stacking from P1 to P2. A lid-like structure formed as a result of a double-strand reversal in the J3/2 bulge partially encloses the SAM binding site and further stabilizes the threeway junction through base-triple interactions that enlarge the major groove of P2. A total of 15 divalent metal ions were identified bound to the S_{MK} RNA to shield the unfavorable electrostatic interactions between sugar phosphate backbones as the result of tertiary RNA folding, FIG. 10 shows the metal binding sites in the S_{MK} box RNA.

The P4 linker helix was further probed by poly(A) scanning mutations. Replacement of the 11-nt hypervariable E. faecalis linker region with a poly(A)₆ sequence did not affect SAM binding activity. However, disrupting the linker helix by further mutating G88 and G89 to adenosine residues dramatically reduced SAM binding (FIG. 6A), consistent with the participation of these residues in the P4 linker helix. Additional point mutations were generated to confirm the formation of the 088-C76 base pair (FIG. 6A). Introduction of a C76U wobble mutation in the E. faecalis S_{MK} box, which occurs naturally in some S_{MK} box family members when a third 77-87 base pair is maintained, resulted in retention of 59% of the SAM binding activity. By contrast, the G88A mismatch mutation that would severely destabilize the linker helix resulted in a 75-fold reduction in SAM binding activity. Combination of the C76U and G88A mutations, which replaces the G-C pair with a weaker A-U pair, restored the binding to 32% of the wild-type level, consistent with sequence alignment results that suggest that an additional base pair is needed in this context to maintain the stability of the linker helix. As shown, the S_{MK} riboswitch, designated S_{mK}6, showed SAM binding activity similar to that of the full-length wild-type construct (FIGS. 6A, 6B).

### Binding-Pocket Formation

The floor of the SAM binding pocket (FIGS. 2A, 2B) is defined by a crucial base-triple interaction (A73•G90-C25), where N1 of A73 approaches from the minor groove side to accept a hydrogen bond from N2 of G90 (FIG. 3A). This base triple serves two purposes: first, it ties J2/4 to the P1 SD-ASD helix in conjunction with another interaction between the N1 of A74 and the 2′-hydroxyl of G90. Furthermore, it orients the N6 amine of A73 for SAM recognition. Base-pairing at C25-G90 is crucial, as a C25•A90 mismatch (G90A mutation) reduced the SAM binding activity to 1.3% of the wild type, whereas a U25-G90 wobble pair (C25U mutation) showed 63% of the SAM binding activity (FIG. 6A). The importance of the A73•G90-C25 base triple is evidenced by the strong, deleterious effect of the C25U G90A double mutation, which converts the SD element from the quintuple-G sequence found in all SMK box elements to the more commonly found GGAGG sequence, while maintaining the SD-ASD pairing. The loss of SAM binding activity in this mutant is apparently due to the loss of N2 when G90 is converted to A, which disrupts the A73•G90 side of the base-triple interaction. Thus, the structural and mutational data collectively explain the absolute conservation of an unusual SD sequence in all SMK box riboswitch RNAs.

The ceiling of the SAM binding site is formed from two layers of nonstandard base pairs, both of which involve bases from the J3/2 trinucleotide bulge, which are highly conserved among the E. faecalis subfamily of the S_{MK} box RNAs. A sheared U72•A64 pair sits directly on top of the SAM intercalation base plane, which exposes the O4 of U72 for SAM recognition. Further above is the A27•G71•G66 base triple, where G71 mediates extensive hydrogen bond contacts to the Hoogsteen face of both A27 and G66, weaving P2 together with J3/2 (FIG. 3B). U65 in the J3/2 bulge mediates a U-turn motif and extends toward the opening of the SAM binding pocket. This residue was shown to be more prone to RNase T1 digestion in the presence of SAM, consistent with the proposal that SAM binding reorients this residue. The backbone of the bulge resembles a ‘lid’ that secludes SAM inside the three-way junction and contributes to the overall electronegative environment inside the pocket, which may help to attract the SAM molecule. The importance of the J3/2 lid structure is indicated by the U65C and U65 deletion mutations, which cause a 5-fold and 20-fold decrease in SAM binding activity, respectively (FIG. 6A).

### SAM Recognition

The universally conserved G26 is left unpaired inside the cavity of the three-way junction. SAM intercalates its adenosine moiety between the P1 and P2 helices from the major groove side, stabilizing the threeway junction through π-stacking interactions (FIG. 3C). The adenosine moiety of SAM adopts an energetically unfavorable syn-conformation and presents its Watson-Crick face to form three minor groove contacts (N1-amino, amino-N3 and amino-2′-OH) with the unpaired G26. Even a minor perturbation of the G26-SAM interaction, such as occurs in the G26A mutant, in which the N1-amino hydrogen bond is disrupted, reduces SAM binding by five-fold (FIGS. 6A and 6B). The N7 at the Hoogsteen face accepts a hydrogen bond from the N6 amine of the tilted A73 (FIG. 3C). Disruption of this hydrogen bond by the A73G mutation caused an 80-fold reduction in SAM binding activity, although this effect cannot be completely separated from the disruption of the A73•G90-C25 base triple. The ribose moiety of SAM adopts a 2′-endo conformation to avoid steric clashes with the base plane of G89 in the SD sequence. The 2′- and 3′-hydroxyl groups of SAM are recognized by hydrogen bonds to the N7 (π-hydrogen bond) and 2′-hydroxyl of G89, respectively (FIG. 3D). The 2′-hydroxyl of SAM donates an additional water-mediated hydrogen bond to the N6 of A74.

The positive charge on the sulfonium ion of SAM is recognized through favorable electrostatic interactions with the O4 carbonyl of U72 and the 2′-hydroxyl of G71 (FIG. 3E). The positive charge is further stabilized through intramolecular electrostatic interactions with the O4′ on the ribose and N3 on the base of SAM (FIG. 3C). Consistent with the structural observations, the U72C mutation, which places a partially positive N4 amine toward the sulfonium, decreases the SAM binding activity by 70-fold (FIG. 6A). A similar charge-stabilization scheme involving contacts between the sulfonium ion of SAM and O4 of uracil is used by the other two classes of SAM binding riboswitches to selectively bind the SAM molecule. The neutral, hydrophobic sulfide in SAH is not expected to make these electrostatic interactions with the RNA.

In contrast to the well-defined adenosine moiety, no electron density was observed for functional groups beyond the sulfonium ion in SAM (FIG. 3C). This is a strong indication that the methionine tail (including the main chain and most of the side chain atoms beyond the sulfur) is not specifically recognized by the SMK box riboswitch, which is in sharp contrast to the extensive recognition of the methionine tail observed in the structures of the S box and SAM-II riboswitches. Lack of recognition toward the methionine tail is further supported by binding studies with SAM analogs, where SAM analogs lacking the methionine main chain atoms bind the S_{MK} box riboswitch with affinities similar to that of SAM. The methyl group on the sulfonium ion is not specified by the S_{MK} box riboswitch (FIG. 3C) because replacing it with an ethyl group does not impair ligand-RNA interactions. The modeled methionine conformation illustrated in FIGS. 2A and 2B complies with all spatial constraints derived from the structural and SAM analog studies.

### Example 3

### Regulation of E. Faecalis metk-lacZ Fusions In Vivo

In this experiment, in vivo lacZ reporter gene assays were conducted as previously described (R. T. Fuchs, et al., 2006, Ibid. and R. T. Fuchs, et al., 2007, Ibid.). Enterococcus faecalis metK leader region constructs, which included the first 15 nt of the coding region, were positioned downstream of the highly expressed B. subtilis glyQS promoter. The resulting DNA fragment was inserted into a lacZ fusion vector (pFG328) to generate an in-frame metK-lacZ translational fusion in which the first five codons of metK were fused to codon 18 of lacZ. The constructs were introduced into the chromosome of B. subtilis strain BR151 (metB10 lys-3trpC2) by integration into an SPβ prophage. Bacillus subtilis strains containing the lacZ fusions were grown at 37° C. in Spizizen minimal medium containing methionine (50 mg ml-l) until early exponential growth. The cells were harvested by centrifugation at 8,000 g and resuspended in Spizizen medium either with or without methionine, and samples were collected at 1-h intervals and assayed for β-galactosidase activity after toluene permeabilization of the cells. All assays were carried out in triplicate and the scientific data are shown in Table 1 below.

TABLE 1
Expression of E. faecalis metK-lacZ fusions

| | −Met | +Met | Ratio |
|---|---|---|---|
| WT | 120 +/− 19 | 24 +/− 5.5 | 5.0 |
| C24G | 110 +/− 14 | 95 +/− 6.1 | 1.2 |
| C24U | 310 +/− 0.71 | 200 +/− 22 | 1.6 |
| C24U + G91A | 23 +/− 2.9 | 15 +/− 2.9 | 1.5 |
| U22G | 220 +/− 27 | 160 +/− 13 | 1.4 |
| A93C | 77 +/− 8.7 | 42 +/− 1.0 | 1.8 |
| U22G + A93C | 11 +/− 3.7 | 2.5 +/− 0.72 | 4.4 |
| C25U | 225 +/− 25 | 80 +/− 5.0 | 2.8 |
| C25U + G90A | 78 +/− 19 | 82 +/− 16 | 0.95 |

The E. faecalis metK S_{MK} box was previously shown to confer translational regulation of a lacZ reporter gene in which the translation-initiation region is replaced by that of the metK gene. Integration of the fusion construct into the B. subtilis chromosome results in high expression when cells are grown under conditions where intracellular SAM pools are low and a five-fold reduction in expression when cells are grown in the presence of high methionine concentrations, conditions that result in high SAM pools (J. Tomsic, et al., J. Bacteriol., 190, 823-833 (2007)).

The S_{MK} box riboswitch structure supports the model that SAM dependent SD-ASD helix formation is responsible for translational inhibition. To verify this hypothesis, the functional consequences of three sets of mutations that perturb the stability of the SD-ASD helix were evaluated. As previously reported (R. T. Fuchs, et al., 2006, Ibid.), a C24G mutation in the ASD region, predicted to disrupt the SD-ASD interaction, causes loss of repression in vivo (Table 1) and loss of SAM binding (FIG. 6A). A C24U mutation had a similar effect (Table 1), although expression was significantly higher and a small response to SAM was retained, presumably because of the maintenance of a U24-G91 wobble pairing. A C24U G91A double mutation, which restores pairing at this position, restored SAM binding (FIG. 6A) and resulted in low expression but only partial restoration of repression by SAM (Table 1). The low expression is likely to be due to disruption of the SD by the G91A mutation, which causes reduced affinity for 30S ribosomal subunits, and therefore, obscures the regulatory response in vivo. A similar pattern was observed with the U22G and A93C mutations (Table 1), which affect the ASD and SD sequences, respectively, and cause loss of SAM binding in vitro (R. T. Fuchs, et al., 2007, Ibid.). The U22G A93C double mutant restores the pairing at this position and restores SAM binding (R. T. Fuchs, et al., 2007, Ibid.). This mutant also showed SAM-dependent repression in vivo, although expression was reduced relative to that of the wild-type construct (Table 1). This reduction in expression may be due to an enhanced SD-ASD interaction from replacing an A-U pair with a G-C pair.

As described above, G90 in the SD sequence has a pivotal role in the S_{MK} box riboswitch, as it organizes the SAM binding pocket through A73•G90-C25 base-triple formation and stacks directly underneath the SAM molecule. The C25U substitution creates a wobble pair with G90. This mutant showed partial repression by high SAM concentrations in vivo (Table 1), consistent with a modest reduction of SAM binding in vitro (FIG. 6A). The C25U G90A double mutant is predicted to restore Watson-Crick pairing at this position but not the crucial base-triple interaction with A73. This variant showed complete loss of repression in vivo, despite maintaining a canonical SD sequence (Table 1), in accordance with the loss of SAM binding in vitro (FIG. 6A). These data confirm the importance of G90 for SAM binding and SAM-dependent regulation.

### Example 4

### Se-SAM-Bound S_{MK} Box Structure Confirms Sulfonium Position

A Se-SAM soaking experiment was used to confirm the sulfonium position in the S_{MK} box riboswitch (i.e., where the sulfur atom was substituted with selenium, a heavier chalcogen analog). To introduce Se-SAM and titrate away bound SAM, S_{MK} crystals were exposed to Se-SAM (10 mM final concentration, freshly prepared and confirmed by mass spectrometry to be intact) during the entire cryoprotection procedure over the course of 2 hours. The crystals were first incubated and then sequentially transferred into three fresh Se-SAM solutions with increasing cryoprotectant concentration and incubated for at least 20 minutes between each transfer before snap-freezing in liquid nitrogen. 2.7 Å diffraction data sets were collected on the APS ID-24E micro diffraction beamline at the K absorption edge of Se. The native S_{MK}6 structure (excluding SAM, metal ions, and water coordinates) was used as the starting model for rigid body and B-factor refinement in Refmac5, followed by simulated annealing refinement in CNS to remove model bias. The structural model was further refined in CNS as described for the SAM-bound structure, and the Se-SAM model was introduced at the later stage of the refinement. The selenium atom in Se-SAM was located in the sigmaA-weighted anomalous difference Fourier map, where the only substantial peak overlaps with the selenium/sulfur position in the structure model. The final Se-SAM-bound S_{MK}6 structure contains fifteen strontium ions and eleven waters with R_{work}/R_{free} of 22.7%/25.5%.

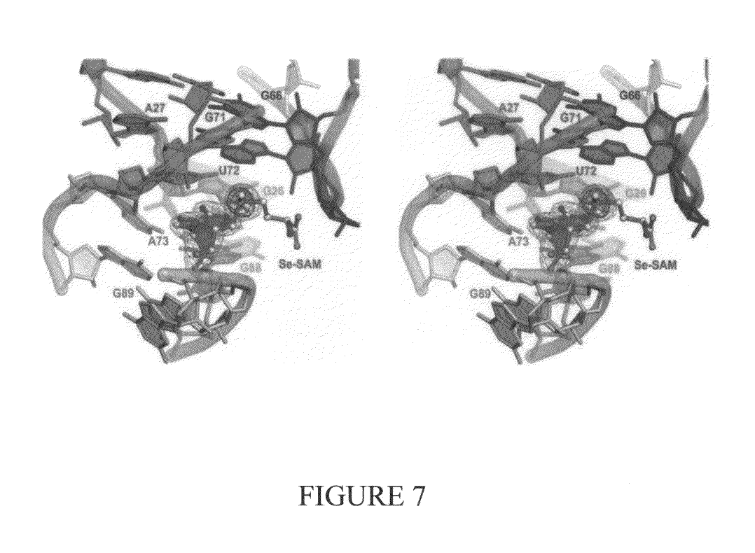

The conformations of the RNA and the Se-SAM molecule are essentially identical to that in the SAM-bound structure, with an r.m.s, deviation of 0.3 Å for all-phosphorous-atom alignment. The same set of contacts specifies Se-SAM in the ligand binding site, and the selenomethionine moiety remains unstructured beyond the onium selenium (FIG. 7). The location of the selenium atom, which essentially overlaps with the sulfur atom in the omit map, is unambiguously identified by an 8σ anomalous difference signal collected at the absorption edge of selenium (FIG. 7). The distances of the electrostatic interactions between the RNA and the onium selenium are on average ˜0.3 Å longer than seen in the SAM-bound structure, reflecting the increased van der Waals radius. Observations from the Se-SAM-bound riboswitch structure confirm the assignment of the sulfonium position in SAM and strengthen the conclusion that the methionine tail of SAM is not specified by the S_{MK} box riboswitch.

### Example 5

### SAH Makes A Minimum Set of Contacts to the S_{MK} Box Riboswitch

To examine the binding of SAH, the S_{MK}6 crystal was repeatedly transferred into solutions containing a saturating amount of SAH (˜2 mM) over the course of three days. The structure was solved using a molecular replacement method from a 2.9 Å data set collected at the microdiffraction beamline 24ID-E at APS. The refinement procedure was essentially the same as described for the Se-SAM structure. Final R_{work}/R_{free} is 22.2%/25.9%. Both structures were verified using simulated annealing omit maps by excluding five bases in each calculation.

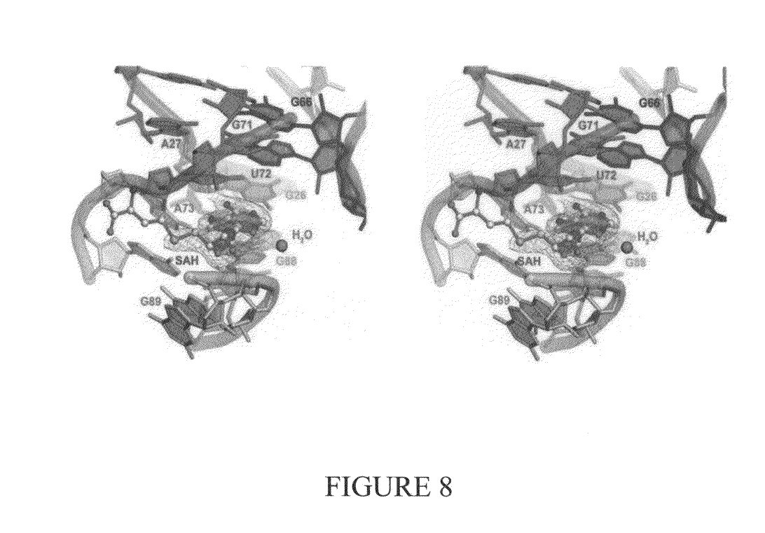

To investigate how the S_{MK} box riboswitch would respond in the presence of natural near-cognate ligands such as SAH, the S_{MK} box RNA structure was determined in the presence of SAH at a saturating concentration of 2 mM, about 67- to 2.000-fold higher than the intracellular concentration of SAH (0.5-30 μM). The resulting structure revealed that SAH can displace SAM at saturating concentration. The binding pocket and overall structure of the S_{MK} box riboswitch remained unchanged, presumably owing to strong crystal lattice contacts to the P1 and P4 helices. The resulting structure is thus a snapshot of the functional SMK box riboswitch sampling a near-cognate ligand before rejecting it. In the 2.9-Å resolution structure, SAH adopts a conformation very different from SAM in the binding pocket (FIG. 8). The ribose moiety of SAH rotates 180°, such that the adenosine moiety now adopts an anti-conformation. 2′- and 3′-hydroxyls in SAH make an alternative set of interactions with the phosphoryl oxygens of G90. More importantly, weak electron-density features revealed that the uncharged sulfide in SAH rotates 180° from U72 to the vicinity of G71. As a result, the intramolecular contacts that stabilize the sulfonium ion conformation in SAM and the key electrostatic interactions between the sulfur atom and the RNA are all lost when SAH is in place. It therefore seems that the positive charge on the sulfur atom is crucial in presenting the SAM molecule in the correct conformation for strong ligand-RNA interactions, analogous to the ‘lock-and-key’ mechanism found in many enzyme-substrate complexes. These structural observations are consistent with the results of in vitro assays that show that the S_{MK} box riboswitch has a much higher affinity for SAM over SAH, with at least a 100-fold preference for SAM.

### Example 6

### In vitro SAM Binding Assays

SAM binding assays were conducted as previously described (R. T. Fuchs et al., 2006 and 2007, Ibid.) with minor modifications. DNA templates corresponding to positions 15-118 (relative to the predicted transcription start site) of the E. faecalis metK leader were constructed by ligating overlapping pairs of complementary oligonucleotides, including a T7 RNA polymerase promoter sequence, as previously described (M. R. Yousef, et al., RNA, 9, 1148-1156 (2003) and B. A., McDaniel, et al., Mol. Microbiol., 57, 1008-1021 (2005)). Ligated products were amplified by PCR and RNAs were synthesized by T7 RNA polymerase transcription using an AmpliScribe T7 High Yield Transcription Kit (Epicentre Biotechnologies). RNAs (3 μM) in 1× transcription buffer (F. J. Grundy, et al., Nucleic Acids Res., 30, 1646-1655 (2002)) were heated to 65° C. for 5 minutes and slow-cooled to 40° C., followed by addition of radiolabeled SAM (3 μM [methyl-3H]-SAM, 15 Ci mmol⁻¹; GE Healthcare) in a total reaction volume of 40 μL, Binding reactions were incubated at room temperature (20-25° C.) for 15 minutes followed by passage through a Nanosep 10K Omega filter (Pall Life Sciences) by centrifugation at 14,000 g for 2.5 minutes. Filters were washed four times with 40 μL 1× transcription buffer to remove unbound SAM, and material retained by the filter was collected, mixed with Packard BioScience Ultima Gold scintillation fluid, and counted in a Packard Tri-Carb 2100TR liquid scintillation counter. No SAM was retained by the filter in the absence of RNA. Hence, the background for non-specific binding was found to be negligible. All binding assays were carried out in triplicate and the scientific data are shown in FIG. 6.

### Example 7

### Apparent K_{d} Determination

The apparent equilibrium dissociation constants (K_{d} values) for the wild-type 15-118 metK leader sequence and the 53-nucleotide crystallization construct were determined using a modified SAM binding assay (J. Tomsic, et al., J. Bacteriol., 190, 823-833 (2007)). Briefly, T7 RNA polymerase transcribed RNA (1 μM) in 1× transcription buffer was refolded as described above and incubated with [³H]-SAM ranging in concentration from 0.02 to 10 μM. Binding reactions were loaded onto Nanosep 3K Omega filters (Pall Life Sciences) and centrifuged briefly at 14,000 g to avoid large volume changes. The concentration of the RNA-[³H]-SAM complex retained by the filter and the unbound SAM present in the flow through were determined by scintillation counting of known volumes, and nonlinear regression analyses were performed using Kaleida-Graph Version 3.51 (Synergy Software). Apparent K_{d} values represent the averages of at least two independent experiments for each construct with a margin of error≦5%.

The apparent K_{d} values for SAM determined by size-exclusion filtration were estimated to be 0.85 μM for the full-length transcript (corresponding to positions 15-118 relative to the predicted E. faecalis metK transcription start site) and 0.57 μM for the 53-nucleotide (nt) crystallization construct (FIG. 6B). In a competition assay, the S_{MK} box riboswitch shows at least 100-fold preference for SAM over its near-cognate ligand SAH (FIG. 6B). The K_{d} value for the full-length S_{MK} transcript was further confirmed by fluorescence-quenching assays, as described below and shown in FIG. 11.

### K_{d} Measurement by 2-AP Fluorescence

To verify the apparent K_{d} value observed in the size-exclusion filtration experiment, a second assay was developed to test the binding affinity of SAM to the wild-type 15-118 metK leader RNA. For this, a bipartite RNA comprised of two half RNAs was used, which corresponds to positions 15-46 and 47-118. The two RNA halves are predicted to anneal through extensive base pairing in the P1-P3 helices to form the full-length metK leader RNA corresponding to positions 15-118, with a nick immediately upstream of residue G47 at the top of the P3 stein loop.

A 2-aminopurine (2-AP) modification was incorporated into the upstream RNA at residue A29 to monitor local conformational changes occurring at this position in response to SAM binding using florescence spectroscopy. In the absence of SAM, sample excitation at 310 nm yielded a fluorescence emission peak at 375 nm. Addition of SAM resulted in a concentration-dependent quenching of 2-AP fluorescence that was not observed with addition of SAH (data not shown). SAM titrations were used to determine an apparent K_{d} value of 1.03 μM for the wild-type metK RNA, which is in close agreement to the value obtained using the size-exclusion filtration assay. No quenching of 2-AP fluorescence was observed with an RNA construct containing a U72C substitution with SAM concentrations up to 100 μM, which is consistent with the loss of SAM binding to the U72C RNA in the filtration assay (FIG. 11).

With the completion of the structure of the S_{MK} box riboswitch, a family portrait of the three known classes of SAM binding riboswitches is now available (for structures of SAM-I and SAM-II, reference is made to R. K. Montange, et al., Nature, 441, 1172-1175 (2006) and S. D. Gilbert, et al., Nat. Struct. Mol. Biol., 15, 177-182 (2008)). These riboswitches adopt completely different RNA folds and have probably emerged independently during evolution. They nevertheless converged at the functional level to preferentially recognize SAM, an important metabolite inside the cell, and regulate gene expression in response to the ligand binding event. It can be concluded from structural comparisons that, although creative ways are used to accommodate the binding of SAM, a conserved mechanism is used by all three classes of SAM riboswitches to distinguish SAM from SAH, which is the biologically relevant SAM analog.

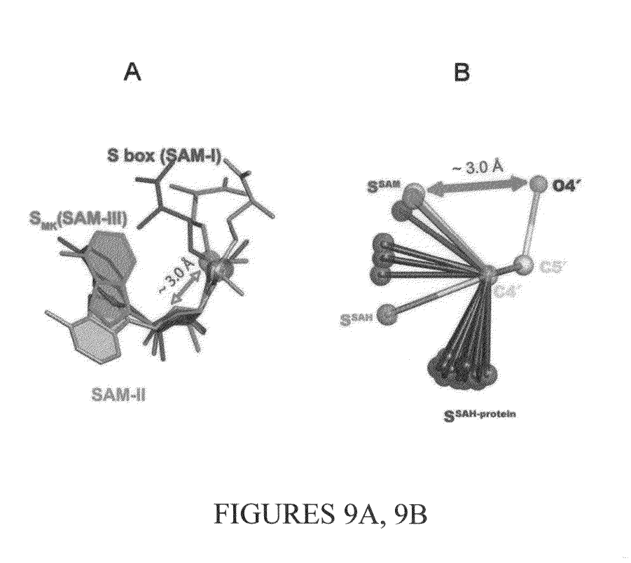

SAM adopts drastically different conformations among the three classes of SAM riboswitches (FIG. 9). The Thermoanaerobacter tengcongensis and B. subtilis yitJ S box (SAM-I) riboswitches completely engulf SAM in a pocket between two helical stacks (R. K. Montage, et al., 2006, Ibid.). The SAM-II riboswitch adopts a classic H-type pseudoknot and encloses SAM inside an RNA triple helix (S. D. Gilbert, et al., 2008, Ibid.). The S_{MK} box riboswitch allows the intercalation of SAM into a tight pocket in a three-way junction. In each case, SAM mediates important tertiary interactions to stabilize the ligand-bound conformation of the riboswitch. The adenine base is involved in base stacking and base-triple interactions in all three riboswitches. The positively charged sulfonium moiety is invariably recognized through favorable electrostatic interactions, usually with one or two O4 carbonyl oxygen atoms from uracil residues. This recognition forms the basis for preferential binding of SAM over SAH. The methyl group on the sulfonium moiety is not directly contacted by any of the three riboswitches. Rather, it points toward the solvent region and is recognized collectively with the sulfonium ion, a mechanism perhaps evolved to prevent self inactivation by spontaneous methylation of the RNA by SAM.

Recognition of the methionine tail, however, occurs differently in the three riboswitches. The tail is extensively contacted by the S box riboswitch, less so by the SAM-II riboswitch, and completely ignored by the S_{MK} box riboswitch. Previous structural studies seem to suggest that riboswitches are more likely to be identified through bioinformatics rather than SELEX approaches because, in each structure, the metabolite is completely encapsulated by the RNA. The ability of the biologically active S_{MK} box riboswitch to recognize SAM by specifying only half of the molecule and without engulfing SAM completely suggests that additional classes of SAM riboswitches can potentially be identified by SELEX approaches using a SAM molecule immobilized at the methionine tail as the bait.

In addition to chemical differences between SAM and SAH, SAM also adopts a distinct conformation that is energetically unfavorable for SAH and other noncognate SAM analogs. Alignment of the conformations of SAM within the three riboswitch structures along the ribose region clearly revealed that, although the conformation of the adenine base (syn- or anti-) and the methionine tail (crouched or extended) varies dramatically among the three riboswitch structures, the sulfonium ion makes an invariable, strong 3-Å electrostatic contact to the O4′ of the SAM ribose (FIGS. 9A and 9B). This gauche-conformation (FIG. 9B) about the C4′-C5′ bond has been shown by NMR measurement to be the predominant (93%) SAM conformation in solution (M. L. Stolowitz, et al., J. Am. Chem. Soc., 103, 6015-6019 (1981)). The same study showed that SAH, on the other hand, favors the anti-conformation, presumably because of its inability to maintain the intramolecular electrostatic interaction. Indeed, in the instant survey of 30 randomly chosen SAH-bound protein structures, 87% of the SAH molecules adopted the anti-conformation (FIG. 9B). Thus, by simultaneously contacting both the ribose and the sulfonium moieties of SAM, all three riboswitches effectively select for a SAM conformation that is unfavorable for SAH, Consistent with this observation, our structural analysis of the SAH-bound S_{MK} box riboswitch revealed that, although SAH could bind to the crystalline-trapped S_{MK} box riboswitch at a nonphysiological high concentration, the sulfide moiety in the riboswitch-bound form of SAH swings 180° away from the sulfonium binding site, severely weakening the ligand-RNA interactions to a level indistinguishable from the binding of other adenosine-containing metabolites such as ATP. Thus, it seems that both chemical and conformational differences at the sugar-sulfur linkage are explored by SAM binding riboswitches to distinguish between cognate and near-cognate ligands. The recent discovery of a new class of SAH binding riboswitches upstream of bacterial genes involved in SAM recycling (J. X. Wang, et al., Mol. Cell., 29, 691-702 (2008)) demonstrates that RNA is capable of forming a selective binding site that favors either SAM or SAH.

A typical riboswitch is conceptually modular, consisting of a ligand-sensing aptamer domain and a separate output domain whose structure is influenced by the ligand binding event at the aptamer domain. A much simpler mechanism for translational regulation is found in the S_{MK} box and SAM-II riboswitches, where the SD sequence is an integral part of the SAM binding aptamer domain, allowing ligand sensing and translational inhibition through SD-sequence sequestration to take place in a single step. An important feature in the S_{MK} box riboswitch is the direct involvement of the SD sequence in SAM recognition. Although strong crystal packing interactions prevent observation of large ligand-induced conformational changes in crystal structures, mutagenesis and enzymatic probing assays clearly revealed global conformational changes in the presence of physiologically relevant concentrations of SAM but not SAH. These SAM-dependent changes are especially evident in the formation of the linker and SD-ASD helices that sequester the ribosome binding site. Conversely, the ability to form the linker and SD-ASD helices is a prerequisite for SAM binding. The combined structural and biochemical data suggest that SAM shifts the conformational equilibrium toward the ligand-bound state seen in the crystal structure, where the SD sequence required for binding of the 30S ribosomal subunit is sequestered. Consistent with this mechanism, ribosome toeprinting analysis showed that the correct positioning of the 30S ribosomal subunit on the S_{MK} box mRNA is reduced in the presence of SAM but not SAH. Direct involvement of the SD sequence in the formation of the aptamer domain is also observed in the SAM-II riboswitch structure, although the exact location of the SD sequence is less well defined.

The combined structural and mutagenesis data on the S_{MK} box riboswitch clearly demonstrate its mechanism for translational inhibition through sequestration of the SD sequence, and the interplay between SD-ASD pairing and SAM binding activity. The S_{MK} box riboswitch is unique among riboswitch RNAs studied to date in that the same residues of the RNA that are involved in gene regulation (the SD sequence) are directly involved in specific recognition of the SAM ligand.

While there have been shown and described what are at present considered the preferred embodiments of the invention, those skilled in the art may make various changes and modifications which remain within the scope of the invention defined by the appended claims.

What is claimed is:

1. A method for identifying a compound that modulates a gene expression of an mRNA molecule containing a S_{MK} box riboswitch that contains a SAM binding pocket, wherein said mRNA molecule is produced by a lactic acid bacteria, the method comprising:
(i) generating a database of structural coordinates that includes atomic positions of the S_{MK} box riboswitch and distances of these atomic positions to atomic positions of a first compound complexed to the S_{MK} box riboswitch;
(ii) identifying key interactions between said S_{MK} box riboswitch and said first compound from said database of structural coordinates; and
(iii) devising a derivative compound of said first compound by use of a computer modeling program, wherein the derivative compound possesses structural modifications, relative to the first compound, that results in an improved modulating interaction between at least one location of the SAM binding pocket and derivative compound.

2. The method of claim 1, further comprising (iv) synthesizing said derivative compound.

3. The method of claim 2, further comprising (v) performing an assay to determine S_{MK} box riboswitch-modulating ability of the derivative compound.

4. The method of claim 2, further comprising synthesizing a second derivative compound of the first compound, wherein the second derivative compound is a further derivative of the first derivative compound, and wherein the second derivative compound possesses structural modifications, relative to the first derivative compound, that results in a further improved modulating interaction between the S_{MK} box riboswitch and second derivative compound.

5. The method of claim 1, wherein the first compound and derivative compound have a chemical structure within the following generic structure: 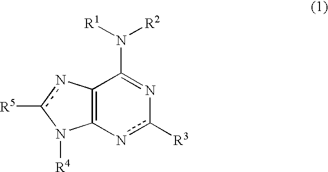
wherein R¹, R², R³, R⁴, and R⁵ are independently selected from hydrogen atom and hydrocarbon groups, wherein said hydrocarbon group is optionally substituted with one or more heteroatoms and/or heteroatom groups; one or both of R³ and R⁵ can additionally be selected from halogen atom, oxo group, and amino group —NR¹R²; R⁴ can additionally be selected from a 4-S-(methionyl)ribosyl group and analogs thereof; and the dashed lines in formula (1) indicate the presence or absence of a double bond.

6. The method of claim 5, wherein R⁴ is represented by the following structure: 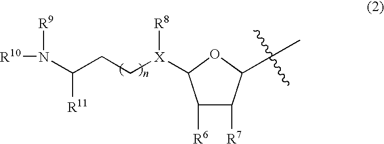
wherein R⁸, R⁹, R¹⁰, and R¹¹ are independently selected from hydrogen atom and hydrocarbon groups, wherein said hydrocarbon group is optionally substituted with one or more heteroatoms and/or heteroatom groups; R¹¹ can additionally be selected from a carboxyl-containing or carboxylester-containing group; R⁶ and R⁷ are selected from hydrogen and hydroxyl groups; X is an oxygen atom, sulfur atom, selenium atom, tellurium atom, nitrogen atom, or ammonium group; and n can be a number of 0, 1, 2, 3, or 4.

7. The method of claim 1, wherein the S_{MK} box riboswitch contains a G-G-G-G-G oligonucleotide sequence in the SAM binding pocket.

8. The method of claim 1, wherein said mRNA molecule containing a S_{MK} box riboswitch is produced by a lactic acid bacteria selected from Streptococcus pneumoniae and Enterococcus faecalis.

9. The method of claim 1, wherein the atomic positions of the S_{MK} box riboswitch included in said database of structural coordinates include at least one atom within a SAM binding pocket of the S_{MK} box riboswitch.

10. The method of claim 9, wherein the at least one atom within the SAM binding pocket of the S_{MK} box riboswitch is within a nucleotide selected from U72, G90, A73, G90, C25, A27, G71, G66, G26, G89, and G71 of the S_{MK} box riboswitch.

11. The method of claim 1, wherein said improved modulating interaction of step (iii) is achieved by computer modeling of a minimum interaction energy, binding energy, or dissociation constant between at least one location of the SAM binding pocket and derivative compound.

12. The method of claim 1, wherein the derivative compound is a compound that deactivates the riboswitch.

13. The method of claim 1, wherein the derivative compound is a compound that functions as an antibiotic against a lactic acid bacteria.

14. The method of claim 13, wherein the antibiotic activity of the derivative compound is evidenced by an ability of the derivative compound to cause inhibition of a translation mechanism of the mRNA containing the S_{MK} box riboswitch.

15. The method of claim 3, wherein said assay is a competition assay between said derivative compound and at least one compound of known binding interaction with the S_{MK} box riboswitch.

16. The method of claim 1, wherein key interactions of step (ii) are further elucidated by employing at least one scanning mutation in the SAM binding pocket of the S_{MK} box riboswitch and comparing the binding interaction between the first or derivative compound and mutated S_{MK} box riboswitch.

17. The method of claim 6, wherein the derivative compound contains at least one fluorescent group such that the derivative compound changes in fluorescence in the presence or absence of a S_{MK} box riboswitch.

18. The method of claim 17, wherein the fluorophore-tagged derivative compound is used in a concentration-dependent fluorescence assay to determine or verify a dissociation constant indicative of the binding energy between the derivative compound and S_{MK} box riboswitch.

19. The method of claim 1, further comprising, before step (i), obtaining a crystal structure of the S_{MK} box riboswitch complexed to the first compound.

20. The method of claim 2, further comprising, after step (ii), obtaining a crystal structure of the S_{MK} box riboswitch complexed to the derivative compound.

21. A method for modulating a gene expression of a lactic acid bacteria, the method comprising contacting said lactic acid bacteria with a derivative compound of claim 5, wherein said derivative compound modulates a gene expression of an mRNA molecule containing a S_{MK} box riboswitch that contains a SAM binding pocket, and wherein said mRNA molecule is within said lactic acid bacteria.

22. The method of claim 21, wherein said derivative compound inhibits gene expression of a lactic acid bacteria.

23. The method of claim 21, wherein said lactic acid bacteria is selected from Streptococcus pneumoniae and Enterococcus faecalis.

24. A derivative compound that modulates a gene expression of an mRNA molecule containing a S_{MK} box riboswitch that contains a SAM binding pocket, the compound having a chemical structure according to claim 5.

25. A derivative compound that modulates a gene expression of an mRNA molecule containing a S_{MK} box riboswitch that contains a SAM binding pocket, the compound having a chemical structure according to claim 6.

26. A method for identifying a riboswitch having a set of structural features in a SAM binding pocket therein that are common with structural features found in a S_{MK} box riboswitch SAM binding pocket, the method comprising entering structural data of said S_{MK} box riboswitch and structural data of at least one riboswitch of interest into a computer database, and instructing the computer to identify those riboswitches that have structural features in common with the structural features of said S_{MK} box riboswitch SAM binding pocket.
